(19) United States
(12) Patent Application Publication
(10) Pub. No.: US 2026/0346424 A1
(43) Pub. Date: Nov. 5, 2026

(54) Perioperative Cobotic System for Asset Management and Surgical Inventory Optimization

(71) Applicant: Peritas AI Inc., Fort Lauderdale, FL (US)

(72) Inventor: Martin Roche, Fort Lauderdale, FL (US)

(73) Assignee: Peritas AI Inc., Fort Lauderdale, FL (US)

(21) Appl. No.: 19/431,214

(22) Filed: Dec. 23, 2025

Related U.S. Application Data
(63) Continuation-in-part of application No. 19/195,731, filed on Apr. 30, 2025.

(51) Int. Cl. B25J 5/00; A61B 34/32

(52) U.S. Cl. CPC B25J 5/007; A61B 34/32

(57) ABSTRACT

A perioperative cobotic system is disclosed for autonomous surgical asset management and inventory optimization. The system includes a mobile collaborative robot configured to navigate perioperative environments while maintaining sterile-field compliance. The cobot includes at least one robotic arm for handling surgical instruments, trays, and supply kits, and an asset identification and tracking module that monitors, in real time, asset location, usage status, and readiness across multiple hospital zones. A predictive inventory optimization engine analyzes historical usage data, surgical schedules, and clinician preferences to recommend inventory replenishment and optimized instrument tray configurations. A control system interfaces with hospital electronic medical record and enterprise resource planning systems to synchronize procedures with instrument availability and dynamically update case carts. Real-time auditing verifies sterilization status and expiration of surgical items, while alerts and user interfaces support operational oversight and workflow efficiency.

### CROSS-REFERENCE TO RELATED APPLICATIONS

This patent application is a continuation-in-part of US Patent Application Ser. No. 19/195,731 filed on Apr. 30, 2025, which claims the benefit of priority to U.S. Provisional Patent Application No. 63/640,524, filed on Apr. 30, 2024, the entirety of which is incorporated herein by reference.

### TECHNICAL FIELD

The present invention relates generally to robotic systems for surgical environments and, more particularly, to a perioperative cobotic system configured for real-time surgical asset tracking, autonomous inventory management, and predictive supply chain optimization.

### BACKGROUND

In modern surgical environments, effective asset management is critical to ensuring procedural efficiency, patient safety, and operational cost control. Surgical teams depend on precise coordination of instruments, implants, and consumables across preoperative, intraoperative, and postoperative phases. However, traditional inventory systems remain fragmented and reactive, often relying on manual tracking, static preference cards, or delayed updates from central processing or electronic medical records (EMRs). These approaches frequently result in inefficiencies such as redundant tray contents, overstocked storage, or case delays caused by missing or unsterilized instruments.

Cobotic systems, collaborative robots designed to operate safely alongside humans, have been introduced in various perioperative roles, but few have been developed to address the full lifecycle of surgical asset management. Existing systems may assist with individual tasks, such as instrument delivery or room turnover, but they typically lack the autonomy, environmental awareness, and integration capabilities needed to manage inventory dynamically in real time.

Furthermore, current solutions rarely include predictive analytics to proactively recommend tray compositions or restocking needs based on upcoming procedures, surgeon preferences, or historical usage trends. Nor do they provide closed-loop verification of instrument readiness, such as real-time auditing of tray completeness, sterilization status, or expiration monitoring for implants and pharmaceuticals. As a result, operating room (OR) staff must manually intervene to validate readiness and respond to anomalies, introducing opportunities for human error and workflow disruption.

There is a clear need for an integrated, autonomous cobotic system that can navigate perioperative environments, identify and track surgical assets in real time, predict and manage inventory needs, and communicate seamlessly with hospital information systems. Such a system would not only reduce inefficiencies and supply redundancies but also enhance safety, standardization, and throughput in surgical operations.

### SUMMARY

The present invention provides a perioperative cobotic system configured for autonomous surgical asset management and inventory optimization across preoperative, intraoperative, and postoperative environments. The system includes a mobile cobot platform capable of autonomously navigating perioperative zones using precision path planning and real-time obstacle avoidance while maintaining sterile field compliance.

The cobot is equipped with at least one robotic arm for handling, organizing, and transporting surgical instruments, trays, and supply kits without human intervention. An integrated asset identification and tracking module utilizes identification technologies, such as RFID, barcode, or computer vision-based recognition, to locate and verify instruments, implants, and disposables in real time. The system continuously monitors the location, usage status, and readiness state of assets across the surgical suite, sterile processing department, and inventory storage areas.

An inventory optimization engine applies machine learning algorithms to analyze historical utilization data, upcoming surgical schedules, and surgeon-specific preferences. Based on this analysis, the system recommends replenishment quantities and instrument tray compositions to reduce redundancy, prevent shortages, and minimize case delays.

A control system governs cobot operation and may optionally interface with electronic medical record (EMR) and enterprise resource planning (ERP) systems. This integration enables dynamic updates to case carts and instrument lists in response to procedural changes or intraoperative tool usage. The system further audits tray completeness, sterilization status, and expiration dates, and issues alerts for missing, contaminated, or expired items.

An interactive user interface displays live dashboards with asset status, location, and usage data. Requests for specific tools or kits may be made via voice, touchscreen, or wearable input. A communication and reporting module logs all asset movements and generates automated utilization and cost-efficiency reports to support perioperative operations management.

The system ensures compliance with surgical safety protocols through real-time situational awareness, sterile field detection, and autonomous adherence to workflow boundaries.

### BRIEF DESCRIPTION OF DRAWINGS

The foregoing and other objects, aspects, features, and advantages of the disclosure will become more apparent and better understood by referring to the following description taken in conjunction with the accompanying drawings, in which:

FIG. 1 is a perspective view of an exemplary perioperative cobotic system.

FIG. 2 is a schematic diagram illustrating an asset identification and tracking system.

FIG. 3 is a block diagram illustrating an inventory optimization engine.

FIG. 4 is a system architecture diagram illustrating integration of the cobotic system control architecture with hospital information systems.

FIG. 5 is a flowchart illustrating a real-time availability and readiness auditing process.

FIG. 6 is an illustration of an interactive user interface of the cobotic system.

FIG. 7 is a flowchart illustrating a communication and reporting method executed by the cobotic system.

FIG. 8 is a communication diagram illustrating data exchange between various systems.

### DETAILED DESCRIPTION

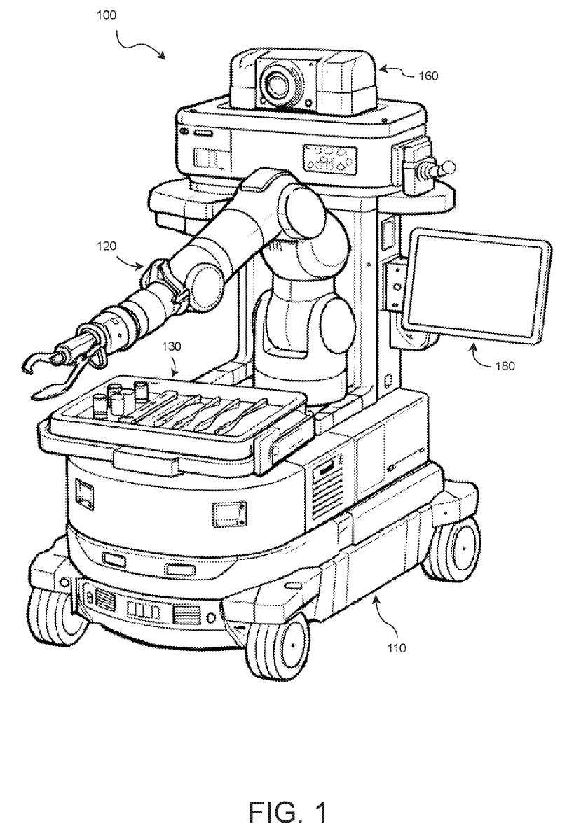

Referring now to the drawings, wherein like reference numerals designate like elements throughout the views, FIG. 1 shows a perioperative cobotic system 100 for asset management and surgical inventory optimization, illustrating an exemplary embodiment of the invention. Cobotic system 100 is a mobile, autonomous platform designed to navigate throughout perioperative environments, including pre-operative areas, operating rooms (intra-operative), and post-operative recovery zones, while maintaining sterile-field compliance through precision path planning and obstacle avoidance. The system 100 includes a mobile base 110, at least one robotic arm 120 mounted on the base, a payload platform 130 for carrying surgical items, an integrated asset identification and tracking system 140, a central control system 150, a suite of environmental and safety sensors 160, a wireless communication module 170, and an interactive user interface 180. The platform is implemented as a collaborative robot (cobot) capable of operating alongside surgical staff without direct human guidance, handling and transporting surgical instruments, instrument trays, and supply kits as needed. In the illustrated embodiment, the base 110 provides locomotion and structural support for the other components, containing the drive mechanism, power supply (e.g. rechargeable batteries), and onboard computing hardware. The robotic arm 120 is a multi-jointed manipulator with an end effector (gripper) configured to securely grasp and release instruments or trays, allowing the cobot to pick up, carry, and position surgical assets autonomously. The base-mounted payload platform 130 (such as a tray table or compartment on the cobot) holds instruments, kits, or sterile trays during transport. The various elements of system 100 are arranged to form a self-contained unit that can move through hospital corridors and operating theaters, deliver required tools to the point of use, and retrieve or organize assets, all while avoiding intrusion into sterile fields and seamlessly integrating into the surgical workflow.

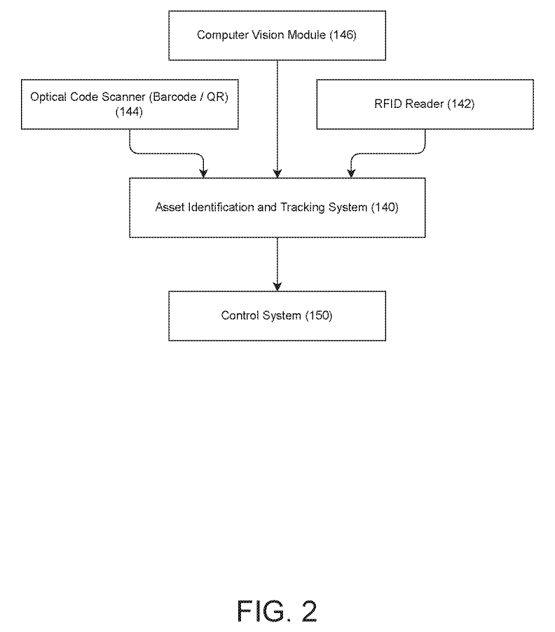

FIG. 2 illustrates the integrated asset identification and tracking system 140 of the cobot and its operation in monitoring surgical assets in real time. Identification and tracking system 140 comprises one or more identification technologies, such as radio-frequency identification (RFID) readers 142, optical code scanners 144, and computer-vision cameras 146, mounted on the cobot (for example, on the arm 120 or the base 110) to automatically recognize and locate surgical items. In use, as the cobot handles an instrument, tray, implant, or disposable supply, the system 140 reads a unique identifier on the item, for instance, scanning an RFID, Rubee, or Bluetooth LE tag or a barcode/QR code affixed to the item, or using vision algorithms to recognize the item by shape or markings. The identification data is passed to the control system 150, which confirms the item’s identity and logs its status and location. The tracking aspect of system 140 continuously monitors each tagged asset’s location and usage status across various hospital zones. For example, an instrument or kit can be detected as it moves from a storage area to a sterile processing department and into an operating room, with system 140 updating a database in real time to reflect that journey. In this manner, the cobot effectively maintains a live inventory map: it knows which instruments are on the surgical field, which are on the cobot’s platform 130, and which are in storage or in transit. The identification and tracking system 140 also verifies asset status before use, for instance, confirming that a scanned instrument is the correct item needed and is authorized or safe for the intended procedure. By employing multiple identification technologies, system 140 ensures robust asset recognition under different conditions (e.g., using RFID for rapid batch detection of many items and computer vision for visual confirmation of specific tools). FIG. 2 thus shows how the cobot’s on-board sensors and readers create an intelligent tracking network that locates and identifies surgical tools and supplies in real time, enabling precise monitoring and management of assets throughout the perioperative process.

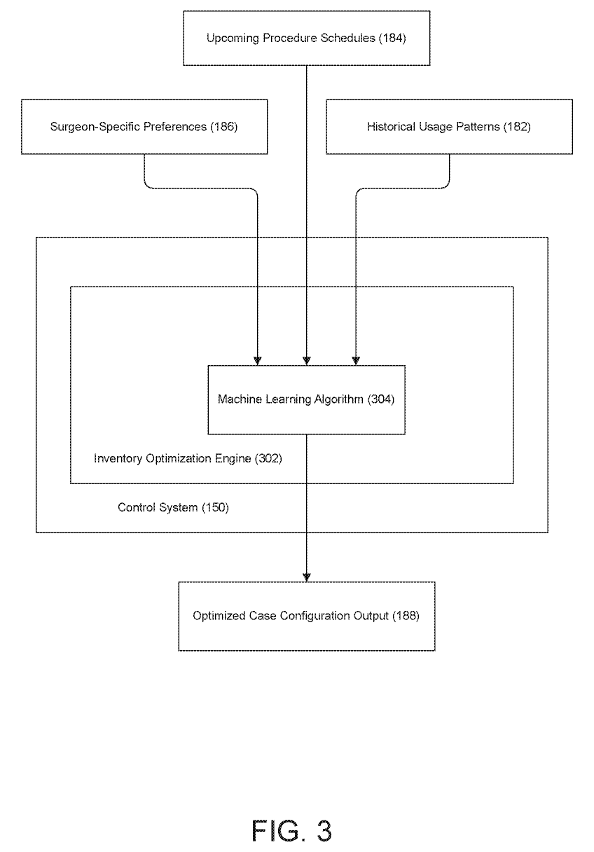

FIG. 3 depicts the inventory optimization engine 302 incorporated into the cobot’s control system 150, which uses predictive modeling to manage surgical inventory efficiently. The inventory optimization engine 302 (executing as a software module within control system 150) analyzes both historical and real-time data to anticipate surgical supply and instrument needs. In the illustrated model, inputs such as historical usage patterns 182 (e.g. data on which instruments and quantities were used in past surgeries of various types), upcoming procedure schedules 184 (information from the hospital’s surgical schedule, including the types of surgeries planned and the preference cards or instrument lists for each surgeon), and surgeon-specific preferences 186 (data on particular tools or implants favored by certain surgeons or known variations in technique) are processed by a machine learning algorithm 304. Using these inputs, the engine predicts the demand for each instrument or supply item for scheduled procedures and even for emergent cases. Based on the predictions, the system 150 can automatically recommend optimal inventory actions: for example, it can suggest the appropriate composition of instrument trays for an upcoming surgery (ensuring all needed tools are included without excess) and calculate recommended replenishment quantities for consumable items (like sutures, sponges, or implants) to avoid stockouts or overstock. The predictions help reduce redundancy, the engine might flag instruments that are consistently unused in certain procedures so they can be omitted from the standard tray, and ensure critical items are not missing (preventing case delays due to a missing tool). In FIG. 3, data flows from the historical and schedule databases into the cobot’s inventory optimization module, which outputs a prepared list or configuration 188 of instruments and supplies for each case. This output may include optimized instrument tray lists, picking or restocking instructions for the cobot (so that it can retrieve necessary kits from storage in advance), and alerts for any anticipated shortages. Through machine learning and continual refinement with each surgery’s data, the inventory optimization engine 302 improves accuracy over time, leading to a highly efficient surgical inventory management process that minimizes waste while guaranteeing availability of required assets.

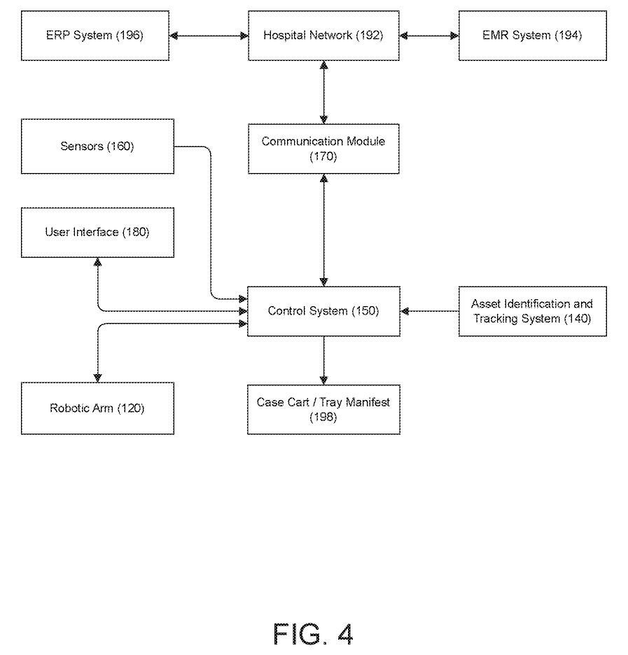

FIG. 4 is a system architecture diagram highlighting the control system 150 of the cobot and its integration with external hospital systems such as electronic medical records (EMR) and enterprise resource planning (ERP) inventory systems. In this schematic, the control system 150 serves as the central “brain” of the cobot, interconnected with all on-board subsystems (the robotic arm 120, identification system 140, sensors 160, communication module 170, user interface 180, etc.) and also linked to hospital networks 192. Through the communication module 170, control system 150 interfaces with the hospital’s EMR system 194 to receive up-to-date surgical schedules, case details, and patient information relevant to inventory (for example, the scheduled procedures and any special instrument requirements noted for those cases). It also connects to the hospital’s ERP or supply chain management system 196 to synchronize inventory data, such as current stock levels of surgical kits or the status of instrument reprocessing. Using these integrations, the cobot can automatically align its operations with the hospital’s workflow: for instance, when a new surgery is scheduled in the EMR, the control system 150 retrieves the list of required instruments and implants and cross-references it with inventory data to ensure those items are available and ready. If a change occurs (for example, a surgery is moved up, canceled, or an additional instrument is requested mid-procedure), the control system dynamically updates the associated instrument list or case cart in coordination with the ERP system. FIG. 4 shows, for example, the control system 150 updating a case cart or tray manifest 198 in real time if an instrument is used unexpectedly during surgery, ensuring that the item is marked for replenishment or sterilization for subsequent procedures. In some embodiments, the control system 150 can even send commands to automated storage or sterile processing equipment (via the ERP interface) to prepare or deliver items. By linking the cobot’s internal decision-making with the hospital’s EMR/ERP infrastructure, the system maintains an up-to-date alignment between surgical plans and inventory status, eliminating manual coordination steps. The control architecture is modular and secure, meaning that if direct integration is not available, the cobot can operate in a standalone mode with pre-loaded schedule data, but when integration is enabled it greatly enhances efficiency by automatically syncing data and responding to changes instantly.

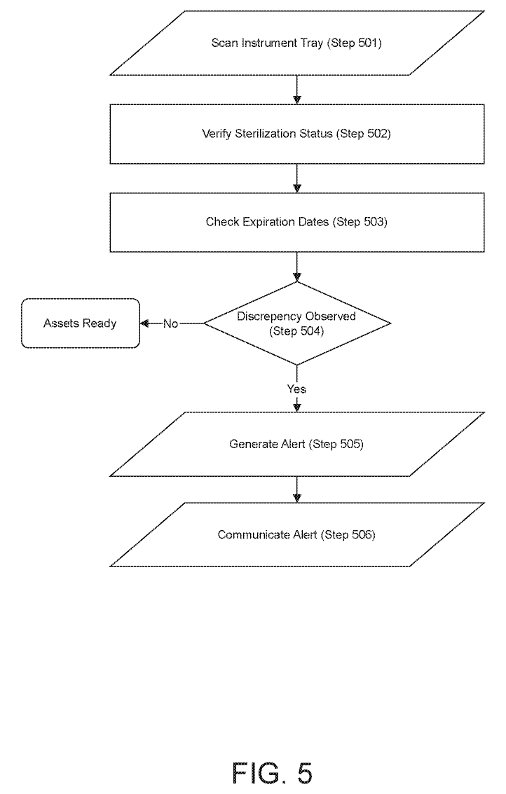

FIG. 5 illustrates the cobot’s real-time availability and readiness auditing functionality for surgical instruments and supplies, implemented as a stepwise flowchart process. The process begins when an audit is triggered, either periodically, event-driven, or upon the cobot handling a tray. Scan Instrument Tray (Step 501) is performed when the identification and tracking system 140, in conjunction with the control system 150, scans all instruments in a tray, for example when the cobot picks up the tray or prior to the start of a surgical procedure, to confirm the presence and identity of each expected asset.

Next, Verify Sterilization Status (Step 502) is executed, in which the control system 150 evaluates sterilization information associated with each scanned instrument, including sterilization indicators or database records confirming that a valid sterilization cycle has been completed within an acceptable timeframe, corresponding to sterilization status verification. The process then proceeds to Check Expiration Dates (Step 503), in which expiration or shelf-life data is evaluated for time-sensitive assets such as implants, medications, or single-use disposables, corresponding to expiration checking.

Following these evaluations, the system performs Determine Asset Readiness (Step 504), in which the control system 150 determines whether any required instrument is missing, incorrect, non-sterile, contaminated, or expired, corresponding to readiness determination. If no discrepancies are detected, the process concludes with the assets confirmed as ready for use. If a discrepancy is detected, the process proceeds to Generate Alert (Step 505), in which an alert is generated in real time identifying the specific issue, such as a missing instrument or expired implant.

Finally, Communicate Alert (Step 506) is performed, in which the generated alert is presented via the user interface 180, such as on the cobot’s display, a connected tablet, or a wearable device, and/or transmitted through the hospital network to notify relevant staff or systems so corrective action may be taken. By executing these steps continuously or at defined checkpoints, the auditing process shown in FIG. 5 ensures that asset readiness issues are detected before instruments are needed in a procedure, thereby improving patient safety, maintaining sterile compliance, and reducing last-minute delays.

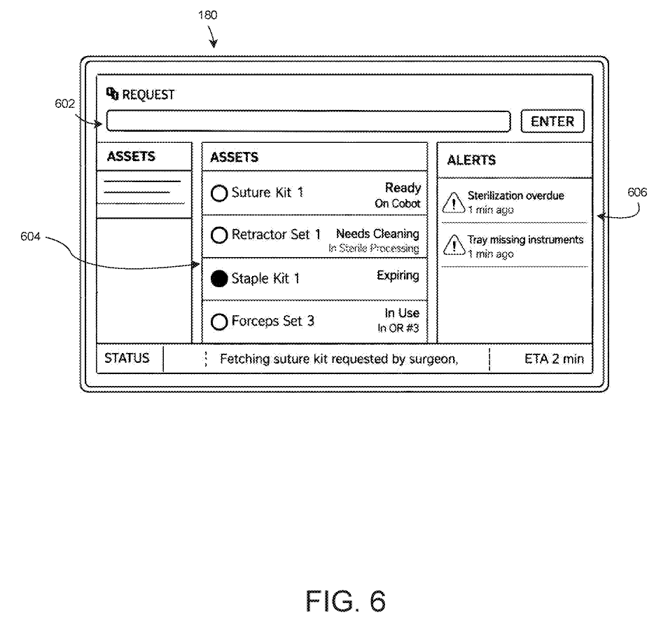

FIG. 6 shows the interactive user interface 180 of the cobotic system, through which operating room staff and sterile processing personnel can engage with the system and monitor inventory status. In one embodiment, the user interface 180 is a display screen and control panel mounted on the cobot (as depicted on base 110 in FIG. 1) that presents a live dashboard 604 of asset information. This dashboard 604 can include visual indicators of each tracked instrument or kit’s location (e.g., “on Cobot,” “in OR #3,” “in Sterile Processing”), usage status (“in use,” “ready,” “needs cleaning”), and any replenishment needs or alerts 606.

FIG. 6 illustrates an example interface layout 180 where assets are listed along with color-coded status icons (for instance, green indicating ready for use, yellow for expiring soon, red for attention needed). The interface 180 is interactive, allowing staff to make requests or inputs 602: for example, an OR nurse can use a touch command to request a specific instrument or supply kit, which triggers the cobot to fetch that item. In addition to the on-board display, the system can accept voice commands as part of the user interface, the cobot may be equipped with a microphone and voice recognition software so that staff can verbally ask for a tool (e.g., “cobot, bring me an extra suture kit”) without breaking sterile technique. The interface may also extend to wearable devices or remote consoles: as shown in FIG. 6, a clinician could use a tablet or a wearable smart device synced to the cobot to view status or send requests from anywhere in the OR or sterile processing department. The control system 150 processes these user requests in real time, dispatching the cobot to fulfill them or updating the display with confirmation and timing (for instance, indicating that a requested item is en route or providing an estimated delivery time). Furthermore, the user interface 180 can display system notifications and guidance, such as alerts generated in FIG. 5’s auditing process, so that personnel are immediately informed of any inventory issues or safety warnings. Overall, FIG. 6 depicts a user-friendly, multimodal interface that enables seamless human-cobot interaction: it provides transparency over asset management operations and empowers surgical staff to summon resources or information on demand via voice, touch, or wearable commands, thereby improving responsiveness and trust in the automated system.

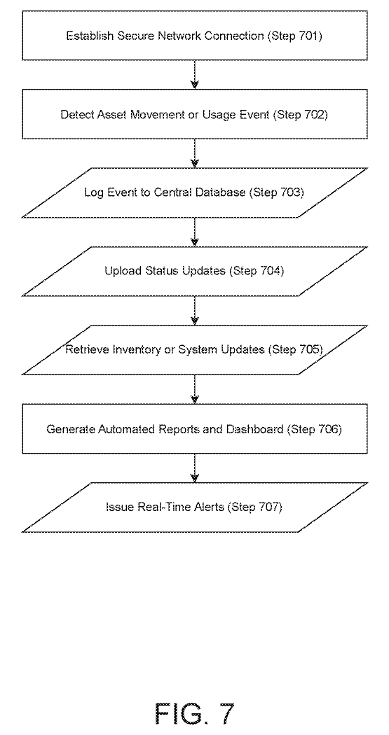

FIG. 7 is a flowchart illustrating a communication and reporting method executed by the cobot system to synchronize operational data with central hospital systems and to support analytics and reporting functions. The method begins with Establish Secure Network Connection (Step 701), in which the cobot’s communication module 170 establishes a secure wireless connection, such as via Wi-Fi or another hospital-approved wireless protocol, between the cobot system 100 and a hospital network.

Next, Detect Asset Movement or Usage Event (Step 702) is performed, in which the cobot detects an asset-related event, such as movement of an instrument tray, use of an instrument during a procedure, or return of an instrument for reprocessing. Upon detection, the process proceeds to Log Event to Central Database (Step 703), in which the communication module 170 transmits event data to a central inventory or asset management database, including identifiers such as asset ID, time, origin, destination, and usage status.

Following logging, the process includes Upload Status Updates (Step 704), in which the cobot uploads ongoing asset status updates to the central database to maintain a real-time inventory record. In parallel, Retrieve Inventory or System Updates (Step 705) may be performed, in which the cobot retrieves current inventory records or receives inbound updates from the central system, such as notifications that newly sterilized kits are available for pickup.

The method further includes Generate Automated Reports (Step 706), in which the communication module 170 compiles usage and performance data into automated reports, such as utilization frequency, tray turnaround times, and inventory consumption metrics, for use by perioperative operations management or supply chain personnel. These reports may be delivered periodically via electronic communication or displayed on a management dashboard 604.

In addition, Issue Real-Time Alerts (Step 707) is performed when a critical condition is detected, such as identification of a missing, expired, or non-sterile instrument. In this step, alert notifications are transmitted to relevant personnel or systems, for example notifying operating room staff and the central inventory system to expedite corrective action.

By executing Steps 701–707, the communication and reporting method shown in FIG. 7 provides a continuous feedback loop that securely logs asset movements, synchronizes inventory data with hospital systems, generates actionable analytics, and supports timely alerts, thereby enhancing traceability, accountability, and data-driven decision-making in perioperative logistics.

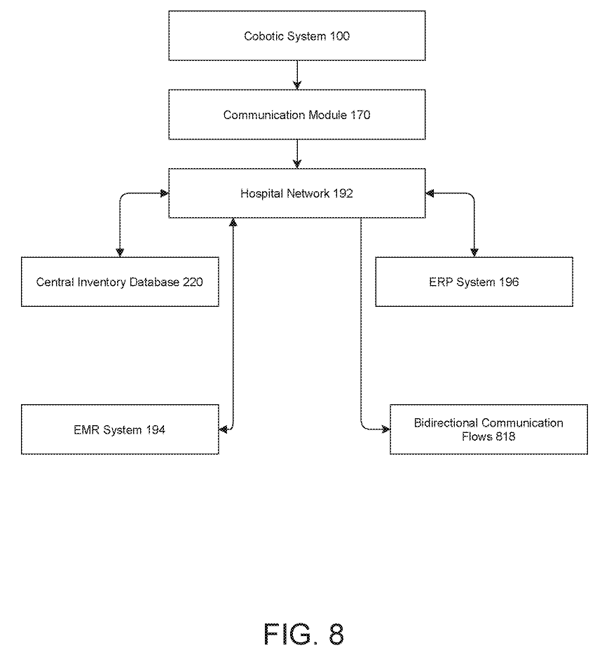

FIG. 8 is a communication diagram illustrating data exchange between the cobotic system 100, central inventory systems, and hospital clinical and administrative platforms. In this embodiment, the cobotic system 100 communicates via its wireless communication module 170 with a hospital network 192, which provides a secure communication infrastructure for exchanging data with multiple external systems.

Through the hospital network 192, the cobotic system 100 exchanges data with a central inventory or asset management database 220, an electronic medical record (EMR) system 194, and an enterprise resource planning (ERP) system 196. The control system 150 coordinates these communications to maintain synchronization between physical surgical assets and digital records maintained by the hospital systems.

Outbound communications from the cobotic system 100 include asset movement events, instrument usage records, sterilization verification results, and inventory consumption data generated by the asset identification and tracking system 140. These data are transmitted as inventory status updates 812 to the central inventory database 220, enabling real-time visibility into instrument locations, tray contents, and supply availability across perioperative areas.

Inbound communications received via the hospital network 192 include scheduled procedure data, case identifiers, and surgeon-specific instrument requirements 814 from the EMR system 194, as well as inventory level information, replenishment confirmations, and sterile processing workflow updates 816 from the ERP system 196. The control system 150 uses this inbound data to anticipate upcoming inventory needs, adjust case cart contents, and synchronize cobot operations with supply chain and reprocessing activities.

FIG. 8 further illustrates bidirectional communication flows 818 between the cobotic system 100 and the external hospital systems, reflecting continuous synchronization of operational, clinical, and inventory data. In some embodiments, alerts generated by the cobotic system—such as detection of a missing, expired, or non-sterile instrument—are transmitted as alert notifications 820 via the hospital network 192 to one or more of the central inventory database 220, EMR system 194, or ERP system 196 to prompt corrective action.

All communications depicted in FIG. 8 are transmitted using encrypted and authenticated protocols in compliance with hospital cybersecurity, privacy, and regulatory requirements. By operating as a network-integrated node within the hospital information ecosystem, the cobotic system 100 maintains real-time alignment between surgical workflows, inventory status, and clinical scheduling data.

While specific embodiments have been described to enable practice of the disclosed subject matter, they are illustrative and not limiting. Variations, modifications, substitutions, and combinations of the features described can be made by those skilled in the art without departing from the scope of this disclosure. Features described with respect to one embodiment may be used with, or substituted for, features of another. Unless expressly stated otherwise, the terms “comprising,” “including,” and “having” are used in an open, non‑limiting sense; the singular forms “a,” “an,” and “the” include the plural forms; and stated ranges include their endpoints and all subranges. Unless the context clearly indicates otherwise, “and/or” means one or more of the listed items (e.g., “A and/or B” encompasses A alone, B alone, or both A and B); “or” is inclusive (i.e., “A or B” means A, B, or both); and “and” is conjunctive. Headings and ordering are for convenience and do not limit the description. The scope of the claimed subject matter is defined solely by the claims that follow and their legal equivalents.

What is claimed is:

1. A perioperative cobotic system for asset management and surgical inventory optimization, comprising:
a mobile cobot platform configured to autonomously navigate perioperative environments, including preoperative, intraoperative, and postoperative zones, using precision path planning and sterile-field avoidance;
at least one robotic arm mounted on the platform and configured to handle and organize surgical instruments, instrument trays, and supply kits; an asset identification and tracking system comprising one or more identification technologies, the asset identification and tracking system being configured to monitor, in real time, locations, usage statuses, and readiness states of surgical assets across multiple hospital zones;
an inventory optimization engine configured to analyze historical surgical instrument usage data and upcoming surgical schedules to predict instrument demand and recommend inventory replenishments and optimized instrument tray compositions;
and a control system integrated with at least one hospital electronic medical record or enterprise resource planning system, the control system being configured to synchronize scheduled surgical procedures with required instrument inventory, dynamically update case cart contents or instrument lists based on changes in surgical plans or instrument usage, and audit instrument readiness.

2. The system of claim 1, wherein the mobile cobot platform comprises environmental sensing components selected from the group consisting of cameras, LiDAR, ultrasonic sensors, or depth sensors, and the platform’s control system is configured to map the environment and detect obstacles and sterile field boundaries to avoid breaching sterile zones during navigation.

3. The system of claim 1, wherein the asset identification and tracking system comprises at least one radio-frequency identification reader, optical code scanner for barcodes or QR codes, or computer vision module to identify and track surgical instruments and supplies.

4. The system of claim 1, wherein the inventory optimization engine employs a machine learning algorithm trained on historical procedure data to forecast upcoming instrument requirements and usage patterns.

5. The system of claim 1, wherein the control system interfaces with the hospital’s electronic medical record or enterprise resource planning systems to retrieve scheduled procedure information and to automatically adjust inventory records and provisioning in real time according to updates in the surgical schedule.

6. The system of claim 1, wherein the control system is configured to dynamically update a case cart or surgical instrument list associated with a scheduled procedure in response to changes in the surgical plan or intraoperative instrument usage.

7. The system of claim 1, wherein the control system issues an alert if a surgical instrument or supply required for an upcoming or ongoing procedure is detected as missing, non-sterile, or expired, thereby prompting staff to take corrective action before it impacts the procedure.

8. The system of claim 1, wherein the system further comprises a user interface including at least one of a display dashboard or a wearable device interface, the user interface being configured to present real-time asset location and status information and to allow perioperative staff to request instruments or supply kits via voice command or touch input.

9. The system of claim 1, wherein the system includes a communication module that wirelessly logs all asset movements, usage events, and inventory changes to a central inventory management database and generates automated utilization and cost-efficiency reports for perioperative operations management.

10. The system of claim 1, wherein the inventory optimization engine prioritizes surgical assets with impending expiration dates or known recall notices by flagging those assets for priority use or replacement in its recommended inventory adjustments.

11. The system of claim 1, wherein the control system is further configured to record the usage of each surgical instrument in association with a particular procedure and to interface with a billing or charge-capture system such that charges for used instruments and supplies are automatically generated or updated in the patient’s billing records.

12. A method for managing perioperative assets and optimizing surgical inventory using a mobile cobot, the method comprising:
autonomously navigating a mobile cobot platform through perioperative areas while avoiding sterile-field zones to position the platform for performing inventory tasks;
identifying and tracking surgical instruments and supplies in the perioperative environment using an asset identification technology on the cobot to obtain real-time location and status data for each asset;
analyzing historical surgical instrument usage data and scheduled surgical procedures to predict upcoming instrument demand and to generate recommended adjustments to instrument inventory or instrument tray configurations;
updating, via a control system integrated with a hospital electronic medical record or enterprise resource planning system, at least one of a case cart content or surgical instrument list based on the predicted instrument demand and on any changes in the surgical schedule or intraoperative instrument usage;
using at least one robotic arm on the cobot to retrieve, transport, or organize surgical instruments, trays, or supply kits in accordance with the generated inventory adjustments or identified surgical needs;
verifying readiness of surgical instruments by checking sterilization status and expiration dates via the asset identification and tracking system, and issuing an alert if any required instrument is found to be missing, contaminated, or expired; and
logging asset usage events and inventory movements in a central database to record instrument utilization and inventory status.

13. The method of claim 12, wherein autonomously navigating the mobile cobot platform comprises scanning the environment with one or more sensors selected from cameras, LiDAR, or ultrasonic sensors to detect obstacles and sterile field boundaries, and dynamically adjusting the cobot’s path to avoid interfering with sterile areas.

14. The method of claim 12, wherein identifying and tracking surgical instruments and supplies comprises reading a unique identifier associated with each item using at least one of an RFID reader to detect an RFID tag, an optical scanner to read a barcode or QR code, or a computer vision system to recognize the item.

15. The method of claim 12, wherein analyzing the historical usage data and scheduled procedures to predict instrument demand includes applying a machine learning model trained on past surgical case data and instrument utilization trends to forecast the instruments and quantities needed for upcoming procedures.

16. The method of claim 12, wherein updating the case cart content or surgical instrument list comprises retrieving procedure schedule information from an electronic medical records system and automatically modifying a corresponding inventory list or database entry in real time to reflect any instrument additions, removals, or substitutions necessitated by changes in the procedure.

17. The method of claim 12, wherein issuing an alert for a missing, contaminated, or expired instrument includes displaying a notification on a user interface accessible via an operating room dashboard or a wearable device, thereby informing surgical staff of the issue and identifying the instrument in need of attention.

18. A perioperative inventory optimization system, comprising: an asset tracking module configured to monitor the location and usage status of surgical instruments and supplies in real time using one or more identification technologies; a predictive inventory management module configured to analyze historical surgical instrument usage data and upcoming surgical schedules to forecast instrument demand and to generate recommended adjustments to instrument inventory levels and instrument tray compositions; and a hospital integration interface configured to receive surgical schedule information from an electronic medical record system and to update corresponding inventory records or case preparation lists based on the recommended inventory adjustments, wherein the system generates an alert if a required surgical instrument for an upcoming procedure is determined to be unavailable or not ready and initiates a replenishment or reprocessing action for that instrument.

19. The system of claim 18, wherein the one or more identification technologies of the asset tracking module include at least one RFID reader, QR code scanner, bar code scanner, and computer vision-based identifier to recognize and track each surgical instrument and supply item.

20. The system of claim 18, wherein the predictive inventory management module employs a machine learning algorithm that incorporates surgeon-specific instrument preferences and historical procedure outcomes to improve the accuracy of demand forecasts and optimize recommended instrument tray compositions.
